(19) United States
(12) Patent Application Publication
(10) Pub. No.: US 2010/0056458 A1
(43) Pub. Date: Mar. 4, 2010

(54) Peptides Inhibiting Angiotensin-Converting Enzyme

(75) Inventors: Jenneke Adriana Cadee, Odijk (NL); Leon Franciscus Mallee, Grubbenvorst (NL)

Correspondence Address: YOUNG & THOMPSON, 209 Madison Street, Suite 500, Alexandria, VA, 22314, US

(73) Assignee: CAMPINA NEDERLAND HOLDING B.V., Zaltbommel (NL)

(21) Appl. No.: 11/994,338

(22) PCT Filed: Jun. 29, 2006
(86) PCT No.: PCT/NL2006/050155

§ 371 Date: Feb. 26, 2008

(30) Foreign Application Priority Data
Jun. 30, 2005 (EP) 05105977.2

(51) Int. Cl. A61K 38/10; C07K 5/06; C07K 5/08; C07K 5/10; C07K 7/06; C07K 7/08; C12P 21/06; A61K 38/05; A61K 38/06; A61K 38/07; A61K 38/08; A23J 1/00

(52) U.S. Cl. 514/14; 530/331; 530/330; 530/329; 530/328; 530/327; 435/68.1; 514/19; 514/18; 514/17; 514/16; 514/15; 426/656

(57) ABSTRACT

The invention provides novel peptides having ACE-inhibiting properties derived from milk proteins, as well as their mixtures and their use as hypotensive aids. The peptides have from 2 up to 14 amino acid residues, at least one of which is a branched-chain amino acid. Examples include IV, LV, VLGPV, VPYPQ, VPYPQR, AVP, KIHP, KYKVPQL, FFVAPFPEVFG, VAPFPEVF, MKGLDI, KGLDI, LR, MHIRLS, MHI, IIA, IAEKT, LKALP, ALP and KK. The invention also provides a process of producing the peptide by enzymatic hydrolysis of milk proteins or other proteins.

This is a 371 National Stage application of international patent application, PCT/NL2006/050155, filed Jun. 29, 2006, which claims priority to European application no. 05105977.2, filed Jun. 30, 2005.

### PEPTIDES INHIBITING ANGIOTENSIN-CONVERTING ENZYME

The invention pertains to novel peptides that are capable of inhibiting angiotensin-converting enzyme (ACE) and to their use in the control and prevention of hypertensia.

### BACKGROUND

Angiotensin-converting enzyme (ACE) converts angiotensin I to angiotensin II, which causes contraction of vascular smooth muscle cells. In addition, ACE deactivates the vasodilator bradykinin. Hence, ACE has a hypertensive effect. Agents capable of inhibiting ACE are thus potential hypotensives. Several peptides are known to have an ACE-inhibiting (ACEI) capacity, some of which are derived from milk-proteins.

U.S. Pat. No. 5,449,661 (Nakamura et al.) discloses the production of the ACEI tripeptide VPP by fermentation of milk by a Lactobacillus helveticus strain JCM 1004. WO 01/032836 and WO 01/032905 (Mäyrä-Mäkinen et al.) describe the production of VPP (SEQ ID No. 122) and IPP (SEQ ID No. 92) by fermentation with L. helveticus strain LBK-16H (DSM 13137). WO 01/034828 (Yamamoto et al.; =EP-A 1 231 279) describes the production of VPP and IPP by a two-step enzymatic proteolysis of casein.

U.S. Pat. No. 6,514,941 (Tolton et al.) describes three peptides having ACEI effect, obtained by trypsin hydrolysis of acid casein. These are denominated C7 (AVPYPQR, (SEQ ID No. 94) (derived from β-casein), and C12 (FFVAPFPEVFGK, SEQ ID No. 95) and C6 (TTMPLW, SEQ ID No. 96) (both derived from α-S2 casein).

EP-A 821 968 (Yamamoto) describes the production of the ACEI dipeptide YP (SEQ ID No. 97) (by fermentation of milk by a Lactobacillus helveticus strain FERM BP-4835.

WO 02/071854 (Draaisma et al.) describes ACEI peptides obtained by fermentation of milk with Lactobacillus delbrueckii lactis, having the β-casein sequences DKIHPF (SEQ ID No. 98) and KVLPVPQ (SEQ ID No. 99) or specific parts thereof.

WO 04/002509 (Tauzin et al.) describes ACEI peptides obtained by trypsin hydrolysis of α-S2 casein, having the sequences TVY (SEQ ID No. 100), NMAINP(SK) (SEQ ID No. 101), FALP(QY(LK)) (SEQ ID No. 102), YL (SEQ ID No. 123), FPQYLQY (SEQ ID No. 103) and ALNEINQFY(QK) (SEQ ID No. 104).

WO 02/019837 (Schlothauer et al.) describes hydrolysis of whey proteins using specific proteases yielding peptides SAP (SEQ ID No. 105), MKG (SEQ ID No. 106), ALPMH (SEQ ID No. 107), LIVTQ (SEQ ID No. 108), LKPTPEGDLEIL (SEQ ID No. 109), INYWL (SEQ ID No. 110) and (LKGYGG)VSLPEW (SEQ ID No. 111).

Van Elswijk et al., J. Chromatography A, 1020 (2003) 45-58, describe the detection and identification of ACEI peptides from milk proteins, and report bioactivity of peptides WLAHK (SEQ ID No. 112), ALPMHIR (SEQ ID No. 113), GLDIQK (SEQ ID No. 114), FALPQYLK (SEQ ID No. 115), FFVAPFPEVFGK (C12, SEQ ID No. 95), AVPYPQR (C7, SEQ ID No. 94), and other ones. WO 04/098310 describes IIAEK (SEQ ID No. 116), IPAVF (SEQ ID No. 117) and IPAVK (SEQ ID No. 118) as ACE inhibiting peptides from β-lactoglobulin.

WO 04/104182 describes bioactive peptides obtained by fermentation of milk with specific strains of Enterococcus faecalis. The peptides include the β-casein-derived peptides LHLPLP (SEQ ID No. 119), VLGPVRPFP (SEQ ID No. 120) and VVVPPF (SEQ ID No. 121).

### DESCRIPTION OF THE INVENTION

Novel peptides having ACE-inhibiting activity were found according to the invention. These novel peptides can be obtained by enzymatic proteolysis of milk fractions, milk proteins or other proteins.

In one embodiment, the invention provides peptides having ACE-inhibiting activity, the peptides having from 2 up to 14 amino acid residues, at least one of which is a branched-chain amino acid (Val, lie, Leu), and being selected from the following sequences IV (SEQ ID No. 1), AIV (SEQ ID No. 2), IIV (SEQ ID No. 3), LIV (SEQ ID No. 4), LV (SEQ ID No. 5), VL (SEQ ID No. 6), VLV (SEQ ID No. 7), LVL (SEQ ID No. 8), LVYP (SEQ ID No. 9), VLGPV (SEQ ID No. 10), VLG (SEQ ID No. 1), LGP (SEQ ID No. 12), GPV (SEQ ID No. 13), VLGP (SEQ ID No. 14), LGPV (SEQ ID No. 15), VLPV (SEQ ID No. 16), VLPVP (SEQ ID No. 17), VLPVPQ (SEQ ID No. 18), LPVPQ (SEQ ID No. 19), VPYPQR (SEQ ID No. 20), VPYPQ (SEQ ID No. 21), AVPY (SEQ ID No. 22), AVPYP (SEQ ID No. 23), VPYP (SEQ ID No. 24), AVP (SEQ ID No. 25), GPFPIIV (SEQ ID No. 26), FPIIV (SEQ ID No. 27), KIHP (SEQ ID No. 28), IHP (SEQ ID No. 29), KYKVPQL (SEQ ID No. 30), YKVP (SEQ ID No. 31), KYKVP (SEQ ID No. 32), FFVAPFPEVFG (SEQ ID No. 33), FVAPFPEVFG (SEQ ID No. 34), FFVAPFPEVF (SEQ ID No. 35), FVAPFPEVF (SEQ ID No. 36), VAPFPEVF (SEQ ID No. 37), HIQK (SEQ ID No. 38), IVP (SEQ ID No. 39), LHSMK (SEQ ID No. 40), MKGLDI (SEQ ID No. 41), KGLDI (SEQ ID No. 42), GLDI (SEQ ID No. 43), LR (SEQ ID No. 44), LRV (SEQ ID No. 45), RVY (SEQ ID No. 46), LRVY (SEQ ID No. 47), MHIRLS (SEQ ID No. 48), MHI (SEQ ID No. 49), IRLS (SEQ ID No. 50), IRL (SEQ ID No. 51), LPMHI (SEQ ID No. 52), LPMHIRL (SEQ ID No. 53), HIRL (SEQ ID No. 54), IRLS (SEQ ID No. 55), IIA (SEQ ID No. 56), IAEKT (SEQ ID No. 57), IIAEKT (SEQ ID No. 58), IAEKTKIP (SEQ ID No. 59), IIAEKTK (SEQ ID No. 60), IIAEKTKIP (SEQ ID No. 61), IAEKTKIP (SEQ ID No. 62), IAEKTKI (SEQ ID No. 63), LKALP (SEQ ID No. 64), KALP (SEQ ID No. 65), ALP (SEQ ID No. 66), ALK (SEQ ID No. 67), DKAL (SEQ ID No. 68), KALPM (SEQ ID No. 69), ALKALP (SEQ ID No. 70), IVTQTM (SEQ ID No. 71), and KGLDIQKVAGTW (SEQ ID No. 72). Also contemplated are peptides having the above sequences with an extension of 1-3 amino acids, especially 1-2, preferably one amino acid, the extension being either at the N-terminus, or, preferably, at the C-terminus, or (if 2 or more amino acids are involved) both.

Preferred peptides from this group comprise IV (SEQ ID No. 1), LV (SEQ ID No. 5), VLGPV (SEQ ID No. 10), VPYPQ (SEQ ID No. 21), VPYPQR (SEQ ID No. 20), AVP (SEQ ID No. 25), KIHP (SEQ ID No. 28), KYKVPQL (SEQ ID No. 30), FFVAPFPEVFG (SEQ ID No. 34), VAPFPEVF (SEQ ID No. 37), MKGLDI (SEQ ID No. 41), KGLDI (SEQ ID No. 42), LR (SEQ ID No. 44), MHIRLS (SEQ ID No. 48), MHI (SEQ ID No. 49), IIA (SEQ ID No. 56), IAEKT (SEQ ID No. 57), LKALP (SEQ ID No. 64) and ALP (SEQ ID No. 66). Most preferred are IV (SEQ ID No. 1), LV (SEQ ID No. 5), AVP (SEQ ID No. 25), and LKALP (SEQ ID No. 64). Especially preferred are the β-casein-derived peptides KK (SEQ ID No. 73), KIHP (SEQ ID No. 28), VPYPQ (SEQ ID No. 21) and VPYPQR (SEQ ID No. 19), and the β-lactoglobulin-derived peptides MKGLDI (SEQ ID No. 41), IAEKT (SEQ ID No. 57), and LKALP (SEQ ID No. 64). These are also individually part of the invention. Another preferred peptide, which can be part of a peptide mixture according to the invention, is the β-lactoglobulin-derived peptide LDIQK (SEQ ID No. 74).

The invention also concerns peptide mixtures containing one or more of the above peptides. In particular, such a peptide mixture contains, on a peptide basis, at least 1 wt. %, preferably at least 3 wt. % especially at least 5 wt. %, of one or more of the individual peptides KK (SEQ ID No. 73), IV (SEQ ID No. 1), LV (SEQ ID No. 5), VLGPV (SEQ ID No. 10), VPYPQ (SEQ ID No. 21), VPYPQR (SEQ ID No. 20), AVP (SEQ ID No. 25), KIHP (SEQ ID No. 28), KYKVPQL (SEQ ID No. 30), FFVAPFPEVFG (SEQ ID No. 34), VAPFPEVF (SEQ ID No. 37), MKGLDI (SEQ ID No. 41), KGLDI (SEQ ID No. 42), LDIQK (SEQ ID No. 74), LR (SEQ ID No. 44), MHIRLS (SEQ ID No. 48), MHI (SEQ ID No. 49), IIA (SEQ ID No. 56), IAEKT (SEQ ID No. 57), LKALP (SEQ ID No. 64) and ALP (SEQ ID No. 66). In particular, such a mixture contains at least 3 wt. %, preferably at least 5 wt. % especially at least 8 wt. %, of one or more of the individual casein-derived peptides KK (SEQ ID No. 73), VPYPQ (SEQ ID No. 21), VPYPQR (SEQ ID No. 19) and KIHP (SEQ ID No. 28). Alternatively or additionally, the mixture contains at least 5 wt. %, especially at least 10 wt. %, or even at least 15 wt. %, of the total of the peptides KK (SEQ ID No. 73), VPYPQ (SEQ ID No. 21), VPYPQR (SEQ ID No. 20) and/or KIHP (SEQ ID No. 28). In another additional or alternative mode, such a mixture contains at least 3 wt. %, preferably at least 5 wt. %, especially at least 8 wt. %, of one or more of the individual β-lactoglobulin-derived peptides MKGLDI (SEQ ID No. 41), LDIQK (SEQ ID No. 74) and LKALP (SEQ ID No. 64). In a particular embodiment, the mixture contains at least 5 wt %, preferably at least 10 wt. %, most preferably at least 15 wt. % of the total of said individual peptides MKGLDI (SEQ ID No. 41), LDIQK (SEQ ID No. 74) and LKALP (SEQ ID No. 64). There is no specific upper limit, but in practice such mixture will not contain more than 50 wt. % of the individual peptides.

A preferred peptide mixtures according to the invention contains AVP (SEQ ID No. 25), LV (SEQ ID No. 5) and IV (SEQ ID No. 1). Another preferred mixtures contains the peptides KIHP (SEQ ID No. 28), KK (SEQ ID No. 73), VPYPQR (SEQ ID No. 20), and KYKVPQ(SEQ ID No. 91). A further preferred peptide mixture contains VLGPV (SEQ ID No. 10), VLPVPQK (SEQ ID No. 75), VPYPQ (SEQ ID No. 21), KK (SEQ ID No. 73), VNELSK (SEQ ID No. 76), and FFVAPFPEVFG (SEQ ID No. 33). Yet another preferred mixture contains the peptides IAEKT (SEQ ID No. 57), LDIQK (SEQ ID No. 74) and LKALP (SEQ ID No. 64). A peptide mixture which is also preferred contains the peptides VLGPV (SEQ ID No. 10), VLPVPQ (SEQ ID No. 18), MKGLDI (SEQ ID No. 41), LR (SEQ ID No. 44), MHIRLS (SEQ ID No. 48) and IIA (SEQ ID No. 56). The preferred amounts of these peptides in the relevant mixture are those as indicated above, i.e. at least 1 wt. % for the individual peptide, and at least 5 wt. % for the particular combination, on a peptide basis.

A particular group of ACE-inhibiting peptides according to the invention are di- and tripeptides containing two adjacent aliphatic amino acids, the N-terminal one of which is selected from Ala, Val, Ile and Leu, and the C-terminal one is selected from Val, Ile and Leu. The optional third (C-terminal) amino acid is preferably one of the neutral small amino acids Gly, Ala and Pro, and more preferably one of the smallest amino acids Gly and Ala, if the N-terminal amino acid is a large amino acid Val, Ile or Leu. Examples thereof include IV (SEQ ID No. 1), LV (SEQ ID No. 5), II (SEQ ID No. 93), IIA (SEQ ID No. 56), ALP (SEQ ID No. 66), AVP (SEQ ID No. 25), VLG (SEQ ID No. 11) and AIV (SEQ ID No. 2). It is preferred that the tripeptide contains from 11 to 15 carbon atoms (G=2, A=3, P, V=5, L, I=6). Tetra- and penta-peptides, comprising this sequence, especially those which are extended at the C-terminus only, in particular with non-basic amino acids, are also contemplated by the invention. Examples include VLGP (SEQ ID No. 14), VLGPV (SEQ ID No. 10), IIAE (SEQ ID No. 77), AVPY (SEQ ID No. 22), IVLN (SEQ ID No. 78), etc.

Another group of active peptides according to the invention is related to the β-casein peptide VLGPV (SEQ ID No. 10). These include shorter homologues thereof (VLG (SEQ ID No. 11), LGP (SEQ ID No. 12), GPV (SEQ ID No. 13), VLGP (SEQ ID No. 14), LGPV (SEQ ID No. 15)), as well as longer homologues up to tetradecapeptides from the β-casein sequence PVLGPVRGPFPIIV (SEQ ID No. 79), for example VLGPVRGPFP (SEQ ID No. 80).

Homologues of the α-S1-casein peptide KYKVPQL (SEQ ID No. 30) that are encompassed by the invention include tri- up to tridecapeptides from the sequence LKKYKVPQLEIVP (SEQ ID No. 81), such as YKVP (SEQ ID No. 31), VPQL (SEQ ID No. 82), VPQLEI (SEQ ID No. 83), PQLEIV (SEQ ID No. 84), but not YKVPQL (SEQ ID No. 85), etc., as well as KK (SEQ ID No. 73).

Homologues of the β-lactoglobulin peptide IIAEKT (SEQ ID No. 58) that are covered by the invention include tri- up to dodecapeptides from the sequence KIIAEKTKIPAVF (SEQ ID No. 86), such as KIIA (SEQ ID No. 87), IA (SEQ ID No. 56), IIAE (SEQ ID No. 77), IAEKT (SEQ ID No. 57), IAEKTKIP (SEQ ID No. 59), etc.

Also covered are the peptide analogues of other mammalian (milk) proteins, where they differ from the bovine peptides, for example (MH)IRLA (SEQ ID No. 88) (goat β-lactoglobulin), instead of (MH)IRLS (SEQ ID No. 48) (bovine), ILP (goat, sheep β-casein)) (SEQ ID No. 89) instead of IPP (SEQ ID No. 92), and VPQR (SEQ ID No. 90) (goat, sheep β-casein) instead of VPYPQR (SEQ ID No. 20) (bovine).

The peptides of the invention can be prepared by specific hydrolysis of milk proteins or other protein source. This can be done using proteolytic enzymes, either as such, or by fermentation with a microorganism containing such enzyme in an active form. Combinations of enzymes and/or microorganisms, either sequential or as a mixture, can also be used. Suitable enzymes include commercially available proteases, such as trypsin, chymotrypsin, Alcalase, (Novozymes), Neutrase (Novozymes) Protease N, Protease P6, Protease A (Amano), Flavourzyme (Novozymes), Promod 25P (Biocatalysts, UK), Multifect Neutral (Genencor), Fungal protease (Bio-Cat Inc, USA), Orientase 90N (Hankyu, Japan), Enzeco Fungal Protease 100A (EDC, USA), Proteinase K, pepsin, papain, thermolysin, elastase, Arg-C proteinase, Asp-N proteinase. Furthermore, besides proteases, (exo)peptidases may be used, e.g. to reduce the bitterness of the hydrolysates obtained, or to remove specific C- or N-terminal amino acids in order to obtain the desired peptides. Examples of peptidases are e.g. leucine aminopeptidases or carboxypeptidases A, B, C and/or Y.

The enzymatic process of the invention can be carried out on milk proteins of any mammal, in particular milk proteins from ungulates, especially ruminants, more in particular members from the family of the Bovidae. The Bovidae include cattle and allies (Bovinae) and goats and allies (Caprinae). Preferred Bovinae species include cattle, yak, buffalo and water buffalo; preferred Caprinae species include sheep and goat. Most preferably the milk protein is bovine milk protein.

In a preferred embodiment, the enzyme reaction is carried out by
1) contacting the protein source with a first protease, the protease comprising a neutral protease;
2) optionally contacting the protein source a second protease or peptidase, or contacting the protein source with a mixture of said first protease and said second protease or peptidase;
3) inactivating and/or separating the proteases and optionally further fractionating and/or purifying the hydrolysate obtained.

The first protease may be a bacterial (e.g. from Bacillus spp.) or fungal neutral protease. Preferably this protease may be selected from the group Neutrase, Protease P6, Protease A, Promod 25P, Multifect Neutral and Fungal Protease.

The second protease or peptidase may be an exoprotease or an endoprotease, or a mixture of the two. Preferred examples of these enzymes include Flavourzyme, Orientase 90N and Enzeco Fungal Protease 100A.

The first protease and the second protease or peptidase may be applied simultaneously or one after the other, with or without intermediate inactivation of the first protease. For example, the hydrolysis may be carried out using the first protease until a degree of hydrolysis of e.g. 3-10%, followed by optional inactivation, and addition of the second protease or peptidase, and further hydrolysis, until the desired total degree of hydrolysis.

The enzymatic reaction is carried out using the conditions which are appropriate for the particular enzyme or enzyme combination. The reaction conditions thus may comprise temperatures between 20 and 60° C., depending on the optimum temperature of the enzymes, pH values e.g. between 3 and 10, depending on the optimum pH of the enzymes, and reaction times that may vary between about 30 min. and 24 h. The reaction times are selected depending on the reaction temperature, and on the concentration, activity and specificity of the enzymes. Generally, reaction conditions are such that a relatively high level of short peptides (less than 12 amino acids) is formed. Degrees of hydrolysis thus may vary, e.g. between 3 and 30%, especially between about 5 and 20%, or even above 7.5 or more preferably at least 10%. When the desired degree of hydrolysis is attained, the hydrolysis reaction can be terminated by separation of the proteolytic enzyme (e.g. in the case of immobilised enzymes), or by inactivation of the enzymes by methods depending on the individual protease, such as acidification to pH 3-4.5, or heating to between 70 and 120° C. for 1 sec to 30 minutes, especially for 5 sec. to 5 min. at between 80 and 100° C.

It was found that the proteolysis medium affects the proteolysis results and the composition of the hydrolysate. Firstly, the level of cations, especially alkali metal and alkaline earth metals, can advantageously be controlled in such a manner that the level of cations from the third period, especially sodium and magnesium, is relatively low, and the level of cations from the fourth period, especially potassium, and calcium, is relatively high.

Thus, during proteolysis, the combined concentration of K and Ca is preferably larger than the combined concentration of Na and Mg, preferably (K+Ca)/(Na+Mg) is at least 2:1 and more preferably between 3:1 and 9:1. Preferably the K+Ca level is between 10 en 500 mM (400 mg-20 g/l), more preferably 15-400 mM, even more preferably 20-300 mM, most preferably 40-200, and especially 50-100 mM. The individual concentrations are preferably 5-400, more preferably 10-200 and most preferably 25-100 mM for K and preferably 5-300, more preferably 10-150 and most preferably 20-75 mM for Ca. Concentrations of Na and Mg are preferably below 30 mM (700 mg/l) and below 20 mM (480 mg/l), respectively.

The presence of the alkali metal and alkaline earth metal cations can be controlled by choosing the appropriate starting material and/or by adding the metals before or during hydrolysis. For example, when preparing casein-derived peptides, it is advantageous to use calcium caseinate as a starting material rather than sodium caseinate. Also, when adjusting the pH before or during hydrolysis, this is preferably done by adding potassium hydroxide or other potassium of calcium-containing salts (e.g. phosphate, citrate, lactate, (bi)carbonate, gluconate), rather than sodium hydroxide or sodium salts.

An additional advantage of the use of potassium and calcium instead of sodium is, that the final peptide or peptide preparation contains a relatively low amount of sodium, which is generally seen as favourable regarding the treatment of hypertension.

Also, during the hydrolysis reaction, a water-miscible cosolvent may be used in addition to water. The amount of cosolvent is preferably between 0.5 and 30 wt %, more preferably between 1 and 20 wt. %, most preferably between 2 and 10 wt. % of the hydrolysis medium. Suitable cosolvents include acetone, alkanols like methanol, ethanol, n-propanol or i-propanol; dimethyl sulfoxide (DMSO); dimethylformamide (DMF); or ethers such as dioxane. The cosolvents may be used alone or in combination with each other.

Another useful hydrolysis method is described in U.S. Pat. No. 6,514,941, e.g. using trypsin as the proteolytic enzyme until an average molar weight below 5000 Da is achieved, followed by ultrafiltration and diafiltration. This procedure can be followed e.g. by Sepharose chromatography.

The hydrolysates obtained can be subjected to further separation using filtration (ultra, nano), chromatography (affinity, ion exchange or other), electrophoresis, extraction, precipitation, and other techniques known in the art, and combinations of such techniques. The final hydrolysate product may be “polished” by filtering over a filter aid, e.g. diatomaceous earth, to remove any insoluble matter.

The individual peptides may also be prepared by chemical synthesis.

When a mixture of active peptides is desired, e.g. skim milk, can be the starting material. The milk may be bovine, goat, sheep, horse, buffalo, yak, human, preferably bovine milk. It is preferred for practical reasons to start with a milk fraction or with a processed, e.g. ultrafiltrated milk, such the salt concentration is reduced. When single peptides are desired, it may be advantageous to start from isolated milk proteins, e.g. isolated or mixed caseinates, whey protein isolates, whey protein concentrates, or isolated whey proteins such as α-lactalbumin or β-lactoglobulin or bovine serum albumin or fractions or partial hydrolysates thereof. When casein is used as a starting material, the serine residues may also be phosphorylated.

Other protein sources are e.g. soy (e.g. yielding an active peptide LR), wheat (including gluten), cottonseed, lupine, pea, potato and other suitable plant proteins; other animal proteins, e.g. egg, blood, meat, fish (bonito, tuna).

It may also be advantageous to perform a post-treatment of the hydrolysates, so as to further diversify or activate the peptide mixture or for debittering the mixture. Such a post-treatment can for example be fermentation with suitable microorganisms, such as lactic acid bacteria, Streptococcus thermophilus, Lactobacillus delbrueckii, Lb. delbrueckii subsp. bulgaricus, Lb. acidophilus, Lb. casei, Lb. helveticus and Bifidobacterium bifidum, or combinations thereof. An example of a suitable treatment comprises fermenting with a yoghurt culture (S. thermophilus and Lb. d bulgaricus). Such a fermentation can be performed on the hydrolysate as such, or on a combination with milk or a milk product. Also other nutrients, such as peptone, tryptone, etc., may be present. The treatment can be performed using conventional conditions for such fermentations, although shorter fermentation periods can also be applied. The ACE inhibiting activity of the product may increase or decrease, but should not decrease with more than 50%, preferably no more than 20% with respect to the starting hydrolysate.

The invention also concerns a food product thus obtainable, i.e. containing at least one of the ACEI peptides described above together with one or more of lactic acid bacteria, especially the bacteria mentioned above.

The identity and the activity of the peptides and their mixtures can be determined by known methods, e.g. as described by Van Elswijk et al. J Chromatography A, 1020 (2003) 45-58, involving liquid chromatography coupled to mass spectroscopy, and on-line bioassay. The active peptides of the invention are characterised by exhibiting an ACE inhibiting concentration (IC₅₀) of below 1500 μM, preferably below 750 μM, more preferably below 250 μM or even below 100 μM. The IC₅₀ can be determined in vitro as described by V. Vermeirsen, J. van Camp & J. Verstraete, “Optimisation and validation of an angiotensin-converting enzyme inhibition assay for the screening of bioactive peptides”. J. Biochem. Biophys. Methods 51, 75-87 (2002). It is noted that peptides having only moderate in vitro activity (relatively high IC₅₀) may become relatively more active in the body as a result of further modification, e.g. splitting off of one or more amino acids.

The invention also pertains to milk protein hydrolysates that are enriched in the ACEI peptides described above. These hydrolysates contain at least 1 wt. % (on dry weight basis) of one or more of the active peptides as defined herein, up to e.g. 50 wt. %. Preferably, the hydrolysates contain between 2 and 40 wt. %, more preferably between 5 and 25 wt. % of the peptides as defined herein. The remainder may be non-active peptides and proteins and/or other active peptides, as well as minor amounts (e.g. below 25 wt. %, preferably between 0.1 and 10 wt. %) of non-proteinaceous material.

The peptides of the invention, as well as their mixtures or mixtures with other peptides are suitable for application in foods, more specifically in functional foods or supplements for the treatment or prevention of hypertension. Functional foods comprise milk or fermented milk products, like yoghurt, beverages, nutrition bars and the like. Optionally, stabilizers (pectin, carragheenan), emulsifiers (mono- and diglycerides), thickeners (alginic acid, chitosan or salts thereof) can be included in the food product. The concentration of the peptides in the food product may be such that between 5 and 500 mg is present per 100 g dry food, or per litre of liquid food. Suitable supplements are e.g. tablets and powders for instant drinks.

The peptides or a specific peptide mixtures may also be suitable for pharmaceutical application to treat or prevent hypertension. The peptides can be administered at a level of between 1-500 mg active peptide/peptide mixture per individual per day. Preferred dosage levels are between 10 and 150 mg/day. In terms of the hydrolysates of the invention containing between 1 and 50 wt/.% of active peptides, these can be administered at a level of between 2 mg and 10 g, depending i.a. on the concentration of active peptides. Preferred dosage levels of the hydrolysates are between 10 mg and 5 g, more preferably between 50 mg and 1 g.

A pharmaceutical composition can comprise the daily dosage level in one dosage unit or in multiple (e.g. 2-6) daily dosage units). The pharmaceutical composition can further contain conventional excipients, stabilisers, flavours, colorants and the like. In particular, the composition may contain an anionic or other polysaccharides as an excipient, for example carboxymethylcellulose, pectin, chitosan, agar, or, especially alginic acid. The anionic polysaccharide may also be in the salt form. The weight ratio between the alginic acid or other polysaccharide and the active peptide may e.g. be between 4:1 and 50:1, especially between 9:1 and 24:1. The pharmaceutical composition may have the form of tablets, capsules, coated tablets, lozenges, syrups, powders, sachets, solutions, suspensions etc. The total weight of the pharmaceutical composition may be in the range of 100-1000 mg of a dry form, e.g. 400-800 mg in case of a tablet.

### EXAMPLES

### Example I

### Preparation of a Protein Hydrolysate Containing Ace Inhibiting Peptides

Calcium caseinate (DMV International, NL) (2.7 kg) was dissolved in water (16 litres), at a temperature of approx. 50° C. An amount of ethanol was added (0.6 litre 50 wt %), and the pH was adjusted to 7.2 using KOH (45 wt %). The total amount of Ca and K was about 90 mM. Neutral Fungal protease from Aspergillus oryzae (Bio-Cat, Inc.) was added in an amount of 0.2% (w/w on protein), and subsequently Flavourzyme (0.1% w/w), and hydrolysis proceeded for 10 hours at 50° C. To terminate the enzyme reaction, a heating step was carried out (10 minutes 90° C.). The solution was then filtered over diatomaceous earth to remove any insoluble particles, and dried to obtain the hydrolysate powder.

The degree of hydrolysis of the product is expressed as the AN/TN ratio (amino nitrogen/total nitrogen)*100. The AN/TN ratio was 12.4. Amino nitrogen is determined according to the Formol titration method. Total nitrogen can be determined using the Kjeldahl method.

### Example II

### Preparation of a Protein Hydrolysate Containing Ace Inhibiting Peptides

A 12% (w/w) solution of WPC 80 (ArlaFoods, Denmark) in water was made, and the pH was adjusted to 7.0 using a 45 wt % KOH solution. An amount of 0.35% Multifect Neutral (bacterial neutral protease, Genencor) was added and the hydrolysis was continued for 6 hours at 50° C. Then the pH was re-adjusted to 6.7 using KOH. The amount of Ca+K was approximately 45 mM.

The reaction mixture was heated to 85° C. for 30 seconds to inactivate the enzyme. The solution was concentrated by evaporation to 20% solids and spray-dried. The AN/TN ratio of the product was 10.

### Example III

### Identification of ACE-Inhibiting Peptides

The peptides obtained according to example I, were analysed using HPLC as described by Van Elswijk et al. J Chromatography A 1020 (2003) 45-58. The HPLC eluate was divided over a mass spectrometry for determining the peptide structure and a bioassay unit for assessing the ACE-inhibiting activity. The following peptides were identified: AVP (SEQ ID No. 25), LV (SEQ ID No. 5) and IV (SEQ ID No. 1). Another batch of the same caseinate contained the peptides KIHP (SEQ ID No. 28), KK (SEQ ID No. 73), VPYPQR (SEQ ID No. 20), and KYKVPQ (SEQ ID No. 91). Starting with other casein sources the peptides VLGPV (SEQ ID No. 10), VLPVPQK (SEQ ID No. 75), VPYPQ (SEQ ID No. 21), KK (SEQ ID No. 73), VNELSK (SEQ ID No. 76), and FFVAPFPEVFG (SEQ ID No. 33) were found.

Similarly the product from example II was analysed and contained IAEKT (SEQ ID No. 57), LDIQK (SEQ ID No. 74) and LKALP (SEQ ID No. 64). Starting from other whey protein sources WPC 60 or whey protein isolate, VLGPV (SEQ ID No. 10), VLPVPQ (SEQ ID No. 18), MKGLDI (SEQ ID No. 41), LR (SEQ ID No. 44), MHIRLS (SEQ ID No. 48), IIA (SEQ ID No. 56) were identified.

### Example IV

### Antihypertensive Composition (Tablets)

| Ingredients | weight % |
|---|---|
| Hydrolysate of example I | 98.8 |
| Magnesium stearate NF FG D | 0.5 |
| (Crompton Corporation) | |
| Stearic Acid Hystrene 5016 NF FG V | 0.5 |
| (Crompton Corporation) | |
| Silicon dioxide Cab-O-Sil M-5 | 0.2 |
| (DMV International GmbH) | |

### Preparation:

The hydrolysate is thoroughly mixed with the silicon dioxide. Magnesium stearate and stearic acid are added, and mixed for 1 minute. Tablets are prepared by direct compression. The tablets are coated with ChromaTone P DDB8876-BL, Chr. Hansen.

### Tableting Parameters:

| | | |
|---|---|---|
| Weight: | 840 | mg |
| Length (oblong): | 19 | mm |
| Thickness: | 6 | mm |
| Compression force: | 18.7 | kN |
| Hardness: | 180 | N |
| Disintegration time: | 11.5 | minutes |

### Example V

### Antihypertensive Food

| Ingredients | Weight % |
|---|---|
| Skimmed milk | 78.0 |
| Water | 11.0 |
| Sugar | 7.0 |
| Maltitol (C*Maltidex 16385, Cerestar) | 3.0 |
| Hydrolysate example II | 0.7 |
| Pectin (Genupectin YM115-L CP Kelco) | 0.3 |
| Pineapple flavour (B80583, Takasago, Japan) | 0.03 |
| Peach flavor (B 80361, Takasago, Japan) | 0.04 |
| Colour (Riboflavine 503065, | 0.001 |
| Sensient Food Colours) | |
| Citrosa (Flavor, NHDC, Exquim S.A., Spain) | 0.0003 |
| Lactic acid | q.s. |
| Culture ABT-2 (Chr. Hansen) | q.s. |

### Preparation:

A stock solution (4%) of pectin in (part of the) water is prepared at 70° C. The milk and the remaining water is mixed, the hydrolysate, sugar, maltitol and citrosa are dissolved in the milk, and the pH is adjusted to 6.7 using lactic acid.

After pasteurisation at 90° C. for 5 minutes, the mixture is cooled to fermentation temperature (37° C.), and the culture is added. Fermentation is continued until the pH is 4.2. The pH is then adjusted to 4.0 using lactic acid. The pectin solution is added, while mixing vigorously. After homogenisation at 120/20 bar at 40° C., the flavours and colour are added and the product is filled into bottles and stored cool.

1. A peptide having ACE-inhibiting activity, the peptide having from 2 up to 14 amino acid residues, at least one of which is a branched-chain amino acid (Val, Ile, Leu), and being selected from the following sequences IV (SEQ ID No. 1), LV (SEQ ID No. 5), VLG (SEQ ID No. 11), VLGP (SEQ ID No. 14), VLGPV (SEQ ID No. 10), VLPV (SEQ ID No. 16), VLPVP (SEQ ID No. 17), VPYPQ (SEQ ID No. 21), VPYPQR (SEQ ID No. 20), AVP (SEQ ID No. 25), FPIIV (SEQ ID No. 27), GPFPIIV (SEQ ID No. 26), KIHP (SEQ ID No. 28), KYKVPQL (SEQ ID No. 30), VAPFPEVF (SEQ ID No. 37), FVAPFPEVF (SEQ ID No. 36), FFVAPFPEVFG (SEQ ID No. 33), HIQK (SEQ ID No. 38), IVP (SEQ ID No. 39), LHSMK (SEQ ID No. 40), MKGLDI (SEQ ID No. 41), LR (SEQ ID No. 44), LRV (SEQ ID No. 45), RVY (SEQ ID No. 46), HIRL (SEQ ID No. 54), MHIRLS (SEQ ID No. 48), IIA (SEQ ID No. 56), IAEKT (SEQ ID No. 57), IIAEKT (SEQ ID No. 58), IAEKTKIP (SEQ ID No. 59), ALK (SEQ ID No. 67), ALP (SEQ ID No. 66), LKALP (SEQ ID No. 64), IVTQTM (SEQ ID No. 71), KGLDIQKVAGTW (SEQ ID No. 72), KK (SEQ ID No. 73), and extended peptides having 1-3 additional amino acids.

2. A peptide having ACE-inhibiting activity, being a dipeptide having the sequences Xaa-Xbb, wherein Xaa is selected from Ala, Val, Ile and Leu, and Xbb is selected from Val, Ile and Leu, or a tripeptide Xaa-Xbb-Xcc, wherein Xaa and Xbb are as above, and Xcc is selected from Gly and Ala and (if Xaa is Ala) Pro, or being a tetrapeptide or pentapeptide comprising the sequence Xaa-Xbb(-Xcc).

3. A peptide according to claim 1, which is selected from IV (SEQ ID No. 1), LV (SEQ ID No. 5), VLGPV (SEQ ID No. 10), VPYPQ (SEQ ID No. 19), VPYPQR (SEQ ID No. 20), AVP (SEQ ID No. 25), KIHP (SEQ ID No. 28), KYKVPQL (SEQ ID No. 30), FFVAPFPEVFG (SEQ ID No. 33), VAPFPEVF (SEQ ID No. 37), MKGLDI (SEQ ID No. 41), KGLDI (SEQ ID No. 42), LR (SEQ ID No. 44), MHIRLS (SEQ ID No. 48), MHI (SEQ ID No. 49), IIA (SEQ ID No. 56), IAEKT (SEQ ID No. 57), LKALP (SEQ ID No. 64), ALP (SEQ ID No. 66) and KK (SEQ ID No. 73).

4. A peptide according to claim 3, which is selected from KK (SEQ ID No. 73), VPYPQ (SEQ ID No. 21), VPYPQR (SEQ ID No. 20), KIHP (SEQ ID No. 28), MKGLDI (SEQ ID No. 41) and LKALP (SEQ ID No. 64).

5. A peptide mixture containing at least one of the peptides according to claim 1.

6. A peptide mixture according to claim 5, which is a milk protein hydrolysate or a combination of two or more hydrolysates containing two or more ACE-inhibiting peptides.

7. A peptide mixture according to claim 5, which contains one or more of the peptides KK (SEQ ID No. 73), VPYPQ (SEQ ID No. 21), VPYPQR (SEQ ID No. 20) and KIHP (SEQ ID No. 28), preferably in an amount of at least 3 wt. % of each of said peptides and/or at least 5 wt % of the total of said peptides, on a peptide basis.

8. A peptide mixture according to claim 5, which contains one or more of the peptides MKGLDI (SEQ ID No. 41), LDIQK (SEQ ID No. 74) and LKALP (SEQ ID No. 64), preferably in an amount of at least 3 wt. % of each of the peptides and/or at least 5 wt. % of the total of said peptides, on a peptide basis.

9. A process of producing one or more peptides having ACE-inhibiting activity, comprising contacting a protein with at least one protease in an aqueous medium in the presence of 40-200 mM of potassium and/or calcium ions, and/or in the presence of 1-20 wt. % of a water-miscible organic solvent, until a degree of hydrolysis of between 3 and 30% is obtained, followed by inactivation or separation of the proteases, and optionally concentrating and/or isolating the peptides obtained.

10. A peptide or mixture of two or more peptides having ACE-inhibiting activity, obtainable by the process of claim 9.

11. A nutritional composition containing one or more peptides according to claim 1, together with at least one nutritional ingredient.

12. A pharmaceutical composition containing one or more peptides according to claim 1, together with at least one pharmaceutically acceptable carrier.

13. (canceled)

14. A milk protein hydrolysate having ACE-inhibiting activity, comprising two or more peptides having from 2 up to 14 amino acid residues, selected from the following sequences IV (SEQ ID No. 1), LV (SEQ ID No. 5), VLG (SEQ ID No. 11), VLGP (SEQ ID No. 14), VLGPV (SEQ ID No. 10), VLPV (SEQ ID No. 16), VLPVP (SEQ ID No. 17), VPYPQ (SEQ ID No. 21), VPYPQR (SEQ ID No. 20), AVP (SEQ ID No. 25), FPIIV (SEQ ID No. 27), GPFPIIV (SEQ ID No. 26), KIHP (SEQ ID No. 28), KYKVPQL (SEQ ID No. 30), VAPFPEVF (SEQ ID No. 37), FVAPFPEVF (SEQ ID No. 36), FFVAPFPEVFG (SEQ ID No. 33), HIQK (SEQ ID No. 38), IVP (SEQ ID No. 39), LHSMK (SEQ ID No. 40), MKGLDI (SEQ ID No. 41), KGLDI (SEQ ID No. 42), LR (SEQ ID No. 44), LRV (SEQ ID No. 45), RVY (SEQ ID No. 46), HIRL (SEQ ID No. 54), MHIRLS (SEQ ID No. 48), MHI (SEQ ID No. 49), IIA (SEQ ID No. 56), IAEKT (SEQ ID No. 57), IIAEKT (SEQ ID No. 58), IAEKTKIP (SEQ ID No. 59), ALK (SEQ ID No. 67), ALP (SEQ ID No. 66), LKALP (SEQ ID No. 64), IVTQTM (SEQ ID No. 71), KGLDIQKVAGTW (SEQ ID No. 72), KK (SEQ ID No. 73), and extended peptides having 1-3 additional amino acids.

15. A milk protein hydrolysate according to claim 14, comprising at least a peptide selected from IV (SEQ ID No. 1), LV (SEQ ID No. 5), VLGPV (SEQ ID No. 10), VPYPQ (SEQ ID No. 21), VPYPQR (SEQ ID No. 20), AVP (SEQ ID No. 25), KIHP (SEQ ID No. 28), KYKVPQL (SEQ ID No. 30), FFVAPFPEVFG (SEQ ID No. 33), VAPFPEVF (SEQ ID No. 37), MKGLDI (SEQ ID No. 41), KGLDI (SEQ ID No. 42), LR (SEQ ID No. 44), MHIRLS (SEQ ID No. 48), MHI (SEQ ID No. 49), IIA (SEQ ID No. 56), IAEKT (SEQ ID No. 57), LKALP (SEQ ID No. 64), ALP (SEQ ID No. 66) and KK (SEQ ID No. 73).

16. A milk protein hydrolysate according to claim 14, comprising at least a peptide selected from KK (SEQ ID No. 73), VPYPQ (SEQ ID No. 21), VPYPQR (SEQ ID No. 20), KIHP (SEQ ID No. 28), MKGLDI (SEQ ID No. 41) and LKALP (SEQ ID No. 64).

17. A peptide mixture having ACE-inhibiting activity, which contains one or more of the peptides KK (SEQ ID No. 73), VPYPQ (SEQ ID No. 21), VPYPQR (SEQ ID No. 20) and KIHP (SEQ ID No. 28), in an amount of at least 3 wt. % of at least one of said peptides and/or at least 5 wt % of the total of said peptides, on a peptide basis.

18. A peptide mixture having ACE-inhibiting activity, which contains one or more of the peptides MKGLDI (SEQ ID No. 41), LDIQK (SEQ ID No. 74) and LKALP (SEQ ID No. 64), in an amount of at least 3 wt. % of at least of the peptides and/or at least 5 wt. % of the total of said peptides, on a peptide basis.

19. A process of producing one or more peptides having ACE-inhibiting activity, comprising contacting a protein with at least one protease in an aqueous medium in the presence of 40-200 mM of potassium and/or calcium ions, and/or in the presence of 1-20 wt. % of a water-miscible organic solvent, until a degree of hydrolysis of between 3 and 30% is obtained, followed by inactivation or separation of the proteases, and optionally concentrating and/or isolating the peptides obtained.

20. A peptide having ACE-inhibiting activity, being a dipeptide having the sequence Xaa-Xbb, wherein Xaa is selected from Ala, Val, Ile and Leu, and Xbb is selected from Val, Ile and Leu, or a tripeptide Xaa-Xbb-Xcc, wherein Xaa and Xbb are as above, and Xcc is selected from Gly and Ala and (if Xaa is Ala) Pro, or being a tetrapeptide or pentapeptide comprising the sequence Xaa-Xbb(-Xcc), said peptide exhibiting an ACE inhibiting concentration (IC₅₀) of below 750 μM.

21. A peptide having ACE-inhibiting activity, the peptide having from 2 up to 14 amino acid residues, at least one of which is a branched-chain amino acid (Val, Ile, Leu), and being selected from the following sequences IV (SEQ ID No. 1), LV (SEQ ID No. 5), VLG (SEQ ID No. 11), VLGP (SEQ ID No. 14), VLGPV (SEQ ID No. 10), VLPV (SEQ ID No. 16), VLPVP (SEQ ID No. 17), VPYPQ (SEQ ID No. 21), VPYPQR (SEQ ID No. 20), AVP (SEQ ID No. 25), FPIIV (SEQ ID No. 27), GPFPIIV (SEQ ID No. 26), KIHP (SEQ ID No. 28), KYKVPQL (SEQ ID No. 30), VAPFPEVF (SEQ ID No. 37), FVAPFPEVF (SEQ ID No. 36), FFVAPFPEVFG (SEQ ID No. 33), HIQK (SEQ ID No. 38), IVP (SEQ ID No. 39), LHSMK (SEQ ID No. 40), MKGLDI (SEQ ID No. 41), LR (SEQ ID No. 44), LRV (SEQ ID No. 45), RVY (SEQ ID No. 46), HIRL (SEQ ID No. 54), MHIRLS (SEQ ID No. 48), IIA (SEQ ID No. 56), IAEKT (SEQ ID No. 57), IIAEKT (SEQ ID No. 58), IAEKTKIP (SEQ ID No. 59), ALK (SEQ ID No. 67), ALP (SEQ ID No. 66), LKALP (SEQ ID No. 64), IVTQTM (SEQ ID No. 71), KGLDIQKVAGTW (SEQ ID No. 72), KK (SEQ ID No. 73).

22. A nutritional composition containing one or more peptides according to claim 20, together with at least one nutritional ingredient.

23. A nutritional composition containing one or more peptides according to claim 21, together with at least one nutritional ingredient.

24. A pharmaceutical composition containing one or more peptides according to claim 20, together with at least one pharmaceutically acceptable carrier.

25. A pharmaceutical composition containing one or more peptides according to claim 21, together with at least one pharmaceutically acceptable carrier.

26. A method for the treatment or prevention of hypertension, comprising administering to a person in need thereof of an ACE inhibiting amount of one or more peptides according to claim 20.

27. A method for the treatment or prevention of hypertension, comprising administering to a person in need thereof of an ACE inhibiting amount of one or more peptides according to claim 21.


SEQUENCE LISTING (123 sequences)

SEQ ID NO: 1 - protein, 2 aa, Bos taurus
IV

SEQ ID NO: 2 - protein, 3 aa, Bos taurus
AIV

SEQ ID NO: 3 - protein, 3 aa, Bos taurus
IIV

SEQ ID NO: 4 - protein, 3 aa, Bos taurus
LIV

SEQ ID NO: 5 - protein, 2 aa, Bos taurus
LV

SEQ ID NO: 6 - protein, 2 aa, Bos taurus
VL

SEQ ID NO: 7 - protein, 3 aa, Bos taurus
VLV

SEQ ID NO: 8 - protein, 3 aa, Bos taurus
LVL

SEQ ID NO: 9 - protein, 4 aa, Bos taurus
LVYP

SEQ ID NO: 10 - protein, 5 aa, Bos taurus
VLGPV

SEQ ID NO: 11 - protein, 3 aa, Bos taurus
VLG

SEQ ID NO: 12 - protein, 3 aa, Bos taurus
LGP

SEQ ID NO: 13 - protein, 3 aa, Bos taurus
GPV

SEQ ID NO: 14 - protein, 4 aa, Bos taurus
VLGP

SEQ ID NO: 15 - protein, 4 aa, Bos taurus
LGPV

SEQ ID NO: 16 - protein, 4 aa, Bos taurus
VLPV

SEQ ID NO: 17 - protein, 5 aa, Bos taurus
VLPVP

SEQ ID NO: 18 - protein, 6 aa, Bos taurus
VLPVPQ

SEQ ID NO: 19 - protein, 5 aa, Bos taurus
LPVPQ

SEQ ID NO: 20 - protein, 6 aa, Bos taurus
VPYPQR

SEQ ID NO: 21 - protein, 5 aa, Bos taurus
VPYPQ

SEQ ID NO: 22 - protein, 4 aa, Bos taurus
AVPY

SEQ ID NO: 23 - protein, 5 aa, Bos taurus
AVPYP

SEQ ID NO: 24 - protein, 4 aa, Bos taurus
VPYP

SEQ ID NO: 25 - protein, 3 aa, Bos taurus
AVP

SEQ ID NO: 26 - protein, 7 aa, Bos taurus
GPFPIIV

SEQ ID NO: 27 - protein, 5 aa, Bos taurus
FPIIV

SEQ ID NO: 28 - protein, 4 aa, Bos taurus
KIHP

SEQ ID NO: 29 - protein, 3 aa, Bos taurus
IHP

SEQ ID NO: 30 - protein, 7 aa, Bos taurus
KYKVPQL

SEQ ID NO: 31 - protein, 4 aa, Bos taurus
YKVP

SEQ ID NO: 32 - protein, 5 aa, Bos taurus
KYKVP

SEQ ID NO: 33 - protein, 11 aa, Bos taurus
FFVAPFPEVFG

SEQ ID NO: 34 - protein, 10 aa, Bos taurus
FVAPFPEVFG

SEQ ID NO: 35 - protein, 10 aa, Bos taurus
FFVAPFPEVF

SEQ ID NO: 36 - protein, 9 aa, Bos taurus
FVAPFPEVF

SEQ ID NO: 37 - protein, 8 aa, Bos taurus
VAPFPEVF

SEQ ID NO: 38 - protein, 4 aa, Bos taurus
HIQK

SEQ ID NO: 39 - protein, 3 aa, Bos taurus
IVP

SEQ ID NO: 40 - protein, 5 aa, Bos taurus
LHSMK

SEQ ID NO: 41 - protein, 6 aa, Bos taurus
MKGLDI

SEQ ID NO: 42 - protein, 5 aa, Bos taurus
KGLDI

SEQ ID NO: 43 - protein, 4 aa, Bos taurus
GLDI

SEQ ID NO: 44 - protein, 2 aa, Bos taurus
LR

SEQ ID NO: 45 - protein, 3 aa, Bos taurus
LRV

SEQ ID NO: 46 - protein, 3 aa, Bos taurus
RVY

SEQ ID NO: 47 - protein, 4 aa, Bos taurus
LRVY

SEQ ID NO: 48 - protein, 6 aa, Bos taurus
MHIRLS

SEQ ID NO: 49 - protein, 3 aa, Bos taurus
MHI

SEQ ID NO: 50 - protein, 4 aa, Bos taurus
IRLS

SEQ ID NO: 51 - protein, 3 aa, Bos taurus
IRL

SEQ ID NO: 52 - protein, 5 aa, Bos taurus
LPMHI

SEQ ID NO: 53 - protein, 7 aa, Bos taurus
LPMHIRL

SEQ ID NO: 54 - protein, 4 aa, Bos taurus
HIRL

SEQ ID NO: 55 - protein, 4 aa, Bos taurus
IRLS

SEQ ID NO: 56 - protein, 3 aa, Bos taurus
IIA

SEQ ID NO: 57 - protein, 5 aa, Bos taurus
IAEKT

SEQ ID NO: 58 - protein, 6 aa, Bos taurus
IIAEKT

SEQ ID NO: 59 - protein, 8 aa, Bos taurus
IAEKTKIP

SEQ ID NO: 60 - protein, 7 aa, Bos taurus
IIAEKTK

SEQ ID NO: 61 - protein, 9 aa, Bos taurus
IIAEKTKIP

SEQ ID NO: 62 - protein, 8 aa, Bos taurus
IAEKTKIP

SEQ ID NO: 63 - protein, 7 aa, Bos taurus
IAEKTKI

SEQ ID NO: 64 - protein, 5 aa, Bos taurus
LKALP

SEQ ID NO: 65 - protein, 4 aa, Bos taurus
KALP

SEQ ID NO: 66 - protein, 3 aa, Bos taurus
ALP

SEQ ID NO: 67 - protein, 3 aa, Bos taurus
ALK

SEQ ID NO: 68 - protein, 4 aa, Bos taurus
DKAL

SEQ ID NO: 69 - protein, 5 aa, Bos taurus
KALPM

SEQ ID NO: 70 - protein, 6 aa, Bos taurus
ALKALP

SEQ ID NO: 71 - protein, 6 aa, Bos taurus
IVTQTM

SEQ ID NO: 72 - protein, 12 aa, Bos taurus
KGLDIQKVAGTW

SEQ ID NO: 73 - protein, 2 aa, Bos taurus
KK

SEQ ID NO: 74 - protein, 5 aa, Bos taurus
LDIQK

SEQ ID NO: 75 - protein, 7 aa, Bos taurus
VLPVPQK

SEQ ID NO: 76 - protein, 6 aa, Bos taurus
VNELSK

SEQ ID NO: 77 - protein, 4 aa, Bos taurus
IIAE

SEQ ID NO: 78 - protein, 4 aa, Bos taurus
IVLN

SEQ ID NO: 79 - protein, 14 aa, Bos taurus
PVLGPVRGPFPIIV

SEQ ID NO: 80 - protein, 10 aa, Bos taurus
VLGPVRGPFP

SEQ ID NO: 81 - protein, 13 aa, Bos taurus
LKKYKVPQLEIVP

SEQ ID NO: 82 - protein, 4 aa, Bos taurus
VPQL

SEQ ID NO: 83 - protein, 6 aa, Bos taurus
VPQLEI

SEQ ID NO: 84 - protein, 6 aa, Bos taurus
PQLEIV

SEQ ID NO: 85 - protein, 6 aa, Bos taurus
YKVPQL

SEQ ID NO: 86 - protein, 13 aa, Bos taurus
KIIAEKTKIPAVF

SEQ ID NO: 87 - protein, 4 aa, Bos taurus
KIIA

SEQ ID NO: 88 - protein, 6 aa, Capra hircus
MHIRLA

SEQ ID NO: 89 - protein, 3 aa, Capra hircus
ILP

SEQ ID NO: 90 - protein, 4 aa, Capra hircus
VPQR

SEQ ID NO: 91 - protein, 6 aa, Bos taurus
KYKVPQ

SEQ ID NO: 92 - protein, 3 aa, Bos taurus
IPP

SEQ ID NO: 93 - protein, 2 aa, Bos taurus
II

SEQ ID NO: 94 - protein, 7 aa, Bos taurus
AVPYPQR

SEQ ID NO: 95 - protein, 12 aa, Bos taurus
FFVAPFPEVFGK

SEQ ID NO: 96 - protein, 6 aa, Bos taurus
TTMPLW

SEQ ID NO: 97 - protein, 2 aa, Bos taurus
YP

SEQ ID NO: 98 - protein, 6 aa, Bos taurus
DKIHPF

SEQ ID NO: 99 - protein, 7 aa, Bos taurus
KVLPVPQ

SEQ ID NO: 100 - protein, 3 aa, Bos taurus
TVY

SEQ ID NO: 101 - protein, 8 aa, Bos taurus
NMAINPSK

SEQ ID NO: 102 - protein, 8 aa, Bos taurus
FALPQYLK

SEQ ID NO: 103 - protein, 7 aa, Bos taurus
FPQYLQY

SEQ ID NO: 104 - protein, 11 aa, Bos taurus
ALNEINQFYQK

SEQ ID NO: 105 - protein, 3 aa, Bos taurus
SAP

SEQ ID NO: 106 - protein, 3 aa, Bos taurus
MKG

SEQ ID NO: 107 - protein, 5 aa, Bos taurus
ALPMH

SEQ ID NO: 108 - protein, 5 aa, Bos taurus
LIVTQ

SEQ ID NO: 109 - protein, 12 aa, Bos taurus
LKPTPEGDLEIL

SEQ ID NO: 110 - protein, 5 aa, Bos taurus
INYWL

SEQ ID NO: 111 - protein, 12 aa, Bos taurus
LKGYGGVSLPEW

SEQ ID NO: 112 - protein, 5 aa, Bos taurus
WLAHK

SEQ ID NO: 113 - protein, 7 aa, Bos taurus
ALPMHIR

SEQ ID NO: 114 - protein, 6 aa, Bos taurus
GLDIQK

SEQ ID NO: 115 - protein, 8 aa, Bos taurus
FALPQYLK

SEQ ID NO: 116 - protein, 5 aa, Bos taurus
IIAEK

SEQ ID NO: 117 - protein, 5 aa, Bos taurus
IPAVF

SEQ ID NO: 118 - protein, 5 aa, Bos taurus
IPAVK

SEQ ID NO: 119 - protein, 6 aa, Bos taurus
LHLPLP

SEQ ID NO: 120 - protein, 9 aa, Bos taurus
VLGPVRPFP

SEQ ID NO: 121 - protein, 6 aa, Bos taurus
VVVPPF

SEQ ID NO: 122 - protein, 3 aa, Bos taurus
VPP

SEQ ID NO: 123 - protein, 2 aa, Bos taurus
YL